(19) United States
(12) Patent Application Publication
(10) Pub. No.: US 2004/0034945 A1
(43) Pub. Date: Feb. 26, 2004

(54) Lightening dye with direct-dyeing colorants

(76) Inventors: Manuela Javet, Marly (CH); Otto Goettel, Marly (CH); Christel Dousse, Courtepin (CH); Hans-Juergen Braun, Ueberstorf (CH)

Correspondence Address: Striker Striker & Stenby, 103 East Neck Road, Huntington, NY, 11743, US

(21) Appl. No.: 10/415,773

(22) PCT Filed: Jul. 13, 2002
(86) PCT No.: PCT/EP02/07813

(51) Int. Cl. A61K 7/13

(52) U.S. Cl. 8/405

(57) ABSTRACT

The object of the invention is a colorant for brightening and coloring keratin fibers which contains (a) an oxidant and (b) at least one yellow direct dye of formula (I) and/or a red direct dye of formula (II) and/or a blue direct dye of general formula (III), and (c) has a basic pH

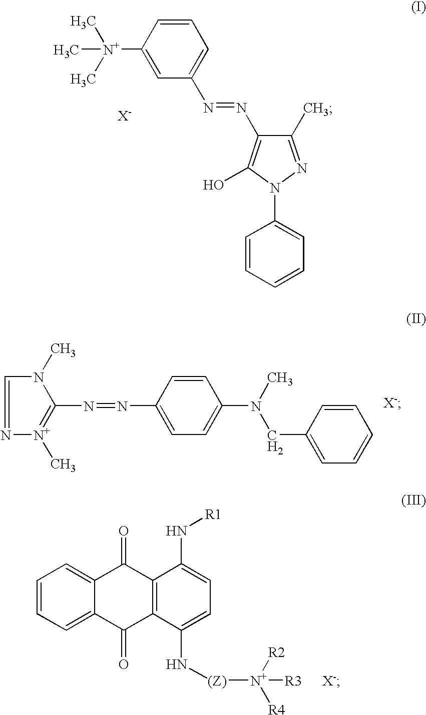

as well as a method for coloring keratin fibers.

The object of the present invention are colorants for brightening and at the same time coloring hair, said colorants containing special direct dyes, and a method for coloring keratin fibers.

Hair colorants are divided essentially into oxidation colorants and nonoxidative toners, depending on the starting color of the hair to be dyed and the desired dyeing result.

Oxidative hair colorants have currently attained major importance. The color in this case is produced by reaction of certain developers with certain couplers in the presence of an appropriate oxidant. The oxidation dyes used for dyeing human hair must meet numerous requirements. For example, these dyes must be physiologically tolerable and produce colorations of the desired intensity. Moreover, the hair colorations obtained must have good light fastness, resistance to permanent waving, acid fastness and rubbing fastness, and under normal conditions they must remain stable for at least 4 to 6 weeks.

Suitable developers are, in particular, 2,5-diaminotoluene, 2,5-diaminophenylethyl alcohol, p-aminophenol and 1,4-diaminobenzene. Frequently used couplers are: resorcinol, 1-naphthol, 3-aminophenol, 5-amino-2-methylphenol, 4-chlororesorcinol and derivatives of m-phenylenediamine. In some persons, the use of these substances can cause intolerance. To avoid allergies, persons who have been sensitized to these substances must avoid them and rely on direct dyes to achieve the desired color shade.

Compared to oxidative colorations, however, the nonoxidative ones as a rule show lower stability and inferior color balance. Moreover, direct colorants as a rule are not capable of “brightening” the hair, because many direct dyes do not tolerate the oxidants needed for brightening and/or the required pH of greater than or equal to 9. The advantages of direct over oxidative colorants lie in a generally lower hair damage, because the direct colorants are normally used at a low pH (lower than 9) and without an oxidant. Direct dyes are often used also as shading aids in oxidative colorants.

In most cases, these are nitro dyes which, consisting of small uncharged molecules, can readily penetrate into the hair, but which because of their size and a lack of charge are also just as readily washed out. Anionic azo dyes are often oxidation-resistant, but, in general, they give sufficiently intense colorations only at an acidic pH value at which it is not possible to achieve brightening at the same time.

In WO 97/20545 are mentioned direct dyes in the yellow and red region which because of their structure and charge are said to provide good adhesion to hair and, because of their stability, permit the simultaneous use of oxidants. A complete coloring system, however, must provide a wide range of different color shades in both the natural and the fashionable color range, namely it must provide yellow, red and blue shades for the fashionable range as well as blond, brown and black shades for the natural range of shades, the natural shades possibly also being produced by mixing different red, yellow and blue dyes.

The purpose of the present invention is therefore to provide a direct dye system that is stable in the basic pH range and is resistant to oxidants—particularly for the yellow, red and blue color range—and which permits the variation of both fashionable and natural shades.

We have now found that this objective can be reached by use of certain direct cationic dyes.

Hence, the object of the present invention is a colorant for brightening and coloring keratin fibers, particularly hair, characterized in that it contains (a) an oxidant and )b) at least one yellow direct dye of formula (I) and/or a red direct dye of formula (II) and/or a blue direct dye of general formula (III) and that (c) it has a basic pH

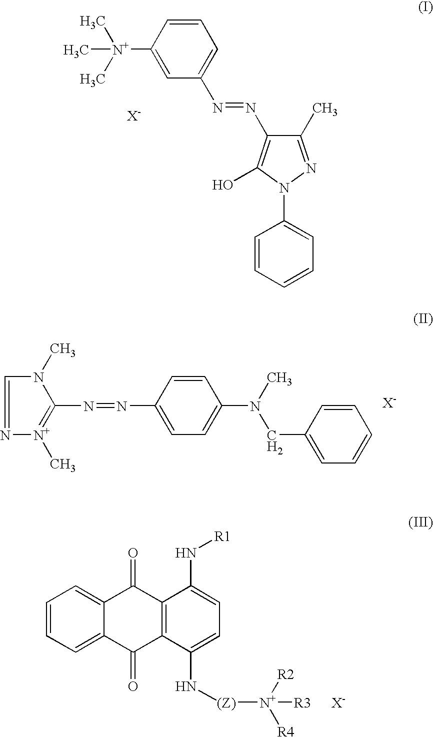

wherein

R1 denotes a hydrogen atom, a (C₁-C₄)-alkyl group, a phenyl group or a (C₂-C₄)-hydroxyalkyl group (particularly a hydroxyethyl group),

R2, R3 and R4 can be equal or different and denote a (C₁-C₄)-alkyl group,

Z denotes a (C₂-C₆)-alkylene diradical and

X⁻ denotes an anion from the group consisting of chloride, bromide, alkylsulfate, arylsulfate, sulfate and acetate.

Preferred dyes of formula (III) are those in which R1 is a (C₁-C₄)-alkyl group, with R2 and R3 denoting a methyl group and R4 denoting a (C₁-C₄)-alkyl group,

Z stands for a C₂- or C₃-alkylene diradical and

X⁻is an anion from the group consisting of chloride, bromide and alkylsulfate.

The dyes of formula (III) can be prepared by a method analogous to that described in European Unexamined Patent Application EP 0 758 547. For example, N,N-dimethyl-3-{[4-(methylamino)-9, 10-diketo-9,10-dihydro-1-anthracenyl]amino}-N-propyl-1-propanaminium chloride or bromide can be prepared as in Example 3 of said EP 0 758 547 but by using propyl chloride or propyl bromide in place of propyl iodide.

The preferred yellow dyes of formula (I) are 3-[(4,5-dihydro-3-methyl-5-keto-1-phenyl-1H-pyrazol-4-yl)azo]-N,N,N-trimethylbenzeneaminium chloride and 3-[(3-methyl-5-hydroxy-1-phenyl-1H-pyrazol-4-yl)azo]trimethylammoniobenzene chloride (Basic Yellow 57), the preferred red dyes of formula (II) being 3 (or 5)-{[4-(benzylmethylamino)-phenyl]azo}-1,2-(or 1,4)-dimethyl-1,2,4-triazolium bromide (C.I. Basic Red 46).

Particularly preferred blue dyes of formula (III) are N,N,N-trimethyl-3-{[4-(methylamino)-9,10-diketo-9, 10-dihydro-1-anthracenyl]amino}-1-propanaminium methyl-sulfate (commercial name: “Astrazone Blue FGLN”), N,N-dimethyl-3-{[4-(methylamino)-9,10-diketo-9,1 0-dihydro-1-anthracenyl]amino}-N-propyl-1-propanaminium chloride and N,N-dimethyl-3-{[4-(methylamino)-9,10-diketo-9,10-dihydro-1-anthracenyl]amino}-N-propyl-1-propanaminium bromide.

The colorant of the invention can contain the dyes of formula (I), (II) or (III) either alone or in admixture with one another, a mixture of at least one dye of formula (III) with at least one dye of formula (I) and/or (II) being particularly preferred.

The colorant of the invention contains the dyes of formulas (I) to (III) in a total amount from about 0.001 to 8 wt. % and preferably from about 0.005 to 4 wt. %.

To extend the color range, the colorant of the invention can contain besides the dyes of formulas (I) to (III) also other natural or synthetic nonoxidative dyes. Examples of natural plant dyes are henna or indigo, and suitable synthetic nonoxidative dyes are azo dyes, triphenylmethane dyes, quinone dyes and particularly nitro dyes, for example 1,4-bis[(2-hydroxyethyl)amino]-2-nitrobenzene; 1-(2-hydroxyethyl)amino-2-nitro-4-[di(2-hydroxyethyl)amino]benzene (HC Blue No. 2); 1-amino-3-methyl-4-[(2-hydroxyethyl)amino]-6-nitrobenzene (HC Violet No. 1); 4-[ethyl-(2-hydroxyethyl)amino]-1-[(2-hydroxyethyl)amino]-2-nitrobenzene hydrochloride (HC Blue No. 12); 4-[di(2-hydroxyethyl)amino]-1-[(2-methoxyethyl)amino]-2-nitrobenzene (HC Blue No. 11); 1-[(2,3-dihydroxypropyl)amino]-4-[methyl-(2-hydroxyethyl)amino]-2-nitrobenzene (HC Blue No. 10); 1-[(2,3-dihydroxypropyl)amino]-4-[ethyl-(2-hydroxyethyl)amino]-2-nitrobenzene hydrochloride (HC Blue No. 9); 1-(3-hydroxypropylamino)-4-[di(2-hydroxyethyl)amino]-2-nitrobenzene (HC Violet No. 2); 1-methylamino-4-[methyl-(2,3-dihydroxypropyl)amino]-2-nitrobenzene (HC Blue No. 6); 2-[(4-amino-2-nitrophenyl)amino]-5-dimethylaminobenzoic acid (HC Blue No. 13); 1 -amino-4-[(2-hydroxyethyl)amino]-2-nitrobenzene (HC Red No. 7); 2-amino-4,6-dinitrophenol, 4-amino-2-nitrodiphenylamine (HC Red No. 1); 1-amino-4-[di(2-hydroxyethyl)amino]-2-nitrobenzene hydrochloride (HC Red No. 13); 1-amino-5-chloro-4-[(2-hydroxyethyl)amino]-2-nitrobenzene; 4-amino-1-[(2-hydroxyethyl)amino]-2-nitrobenzene (HC Red No. 3); 4-amino-3-nitrophenol; 4-[(2-hydroxyethyl)amino]-3-nitrophenol, 1-[(2-aminoethyl)-amino]-4-(2-hydroxyethoxy)-2-nitrobenzene (HC Orange No. 2); 4-(2,3-dihydroxypropoxy)-1-[(2-hydroxyethyl)amino]-2-nitrobenzene (HC Orange No. 3); 1-amino-5-chloro-4-[(2,3-dihydroxypropyl)amino]-2-nitrobenzene (HC Red No. 10); 5-chloro-1,4-[di(2,3-dihydroxypropyl)amino]-2-nitrobenzene(HC Red No. 11); 2-[(2-hydroxyethyl)amino]-4,6-dinitrophenol; 4-ethylamino-3-nitrobenzoic acid; 2-[(4-amino-2-nitrophenyl)amino]benzoic acid, 2-chloro-6-ethylamino-4-nitrophenol; 2-amino-6-chloro-4-nitrophenol; 4-[(3-hydroxypropyl)amino]-3-nitrophenol; 2,5-diamino-6-nitropyridine; 1,2,3,4-tetrahydro-6-nitroquinoxaline; 7-amino-3,4-dihydro-6-nitro-2H-1,4-benzoxazine (HC Red No. 14); 1-amino-2-[(2-hydroxyethyl)amino]-5-nitrobenzene (HC Yellow No. 5); 1-(2-hydroxyethoxy)-2-[(2-hydroxyethyl)amino]-5-nitrobenzene (HC Yellow No. 4); 1-[(2-hydroxyethyl)amino]-2-nitrobenzene (HC Yellow No. 2); 2-[(2-hydroxyethyl)amino]-1-methoxy-5-nitrobenzene;2-amino-3-nitrophenol; 1-(2-hydroxyethoxy)-3-methyl-amino-4-nitrobenzene; 2,3-(dihydroxypropoxy)-3-methylamino-4-nitrobenzene, 2-[(2-hydroxyethyl)amino]-5-nitrophenol (HC Yellow No. 11); 3-[(2-aminoethyl)amino]-1-methoxy-4-nitrobenzene hydrochloride (HC Yellow No. 9); 1-[(2-ureidoethyl)amino]-4-nitrobenzene; 4-[(2,3-dihydroxypropyl)amino]-3-nitro-1-trifluoromethylbenzene (HC Yellow No. 6); 1-chloro-2,4-bis[(2-hydroxyethyl)amino]-5-nitrobenzene (HC Yellow No. 10); 4-[(2-hydroxyethyl)-amino]-3-nitro-1-methylbenzene, 1-chloro-4-[(2-hydroxyethyl)amino]-3-nitrobenzene (HC Yellow No.12); 4-[(2-hydroxyethyl)amino]-3-nitro-1-trifluoromethylbenzene (HC Yellow No. 13); 4-[(2-hydroxyethyl)amino]-3-nitrobenzonitrile (HC Yellow No. 14) and 4-[(2-hydroxyethyl)amino]-3-nitrobenzamide (HC Yellow No. 15).

Moreover, besides the dyes of formulas (I) to (III), it is also possible to use cationic azo dyes, for example 1,4-dimethyl-5-{[4-(dimethylamino)phenyl]azo}-1,2,4-triazolium chloride (C.I. 11055; Basic Red 22), 1-methyl-4-{[methyl(phenyl)hydrazono]methyl}pyridinium chloride (Basic Yellow 87), 1-methyl-4-{(E)-[methyl(4-methoxyphenyl)hydrazono]methyl}pyridinium chloride, 1-methyl-4-{[methyl(4-methoxyphenyl)hydrazono]methyl}pyridinium methylsulfate (Basic Yellow 91), 2-{[4-(dimethylamino)-phenyl]azo}-1,3-dimethyl-1H-imidazol-3-ium chloride (Basic Red 51), 5-{[4-(dimethylamino)phenyl]azo}-1,2-dimethyl-1H-pyrazol-2-ium chloride,1,3-dimethyl-2-{[4-(methylamino)phenyl]azo}-1H-imidazol-3-ium chloride (Basic Red 109), 2-[(4aminophenyl)azo]-1.3-dimethyl-1H-imidazol-3-ium chloride, 4-{[4-(dimethylamino)phenyl]azo}-1-methylpyridinium chloride and N,N-dimethyl-4-[(E)-(1-oxido-4-pyridinyl)diazenyl]aniline.

In special cases, depending on the dye carrier composition employed, it is possible to add anionic (“acidic”) dyes compatible with the cationic dyes used.

The colorant of the invention contains a total amount of natural and/or synthetic nonoxidative dyes from about 0.01 to 15 wt. % and particularly from about 0.1 to 12 wt. %.

Of course, it is also possible to add to the colorant of the invention an oxidative dye precursor, for example a paraphenylenediamine, metaphenylenediamine, aminophenol or 4,5-diaminopyrazole.

Each of the additional developers and couplers is contained in the colorant in a total amount from 0.01 to 20 wt. %, preferably from about 0.1 to 10 wt. % and particularly from 0.1 to 5 wt. %.

To increase the color intensity, a carrier commonly used in cosmetic systems can be added, if necessary. Suitable compounds are described, for example, in German Unexamined Patent Application DE 196 18 595, the disclosure of which, in particular, is hereby included by reference. Particularly well suited carriers are, for example, benzyl alcohol, vanillin and isovanillin.

For coloring, the aforedescribed dyes are applied from a suitable dye carrier composition.

The colorant of the invention can be formulated to be in the form of, for example, a solution, particularly an aqueous or aqueous-alcoholic solution. A particularly preferred form, however, is a cream, gel or emulsion. Such compositions consist of a mixture of dyes with additives commonly used for such preparations.

Common additives to solutions, creams, emulsions or gels are, for example, solvents such as water, lower aliphatic alcohols, for example ethanol, propanol or isopropanol, glycerol or glycols such as 1,2-propylene glycol; moreover wetting agents or emulsifiers from the classes of anionic, cationic, amphoteric or nonionic surface-active substances such as, for example, the fatty alcohol sulfates, ethoxylated fatty alcohol sulfates, alkylsulfonates, alkylbenzenesulfonates, alkyltrimethylammonium salts, alkylbetaines, ethoxylated fatty alcohols, ethoxylated nonylphenols, fatty alkanolamides, ethoxylated fatty esters, furthermore thickeners such as the higher fatty alcohols, starch or cellulose derivatives, petrolatum, paraffin oil, fatty acids; moreover hair-care agents such as cationic resins, lanolin derivatives, cholesterol, pantothenic acid and betaine. The said constituents are used in amounts commonly employed for such purposes, for example the wetting agents and emulsifiers at a concentration from about 0.1 to 30 wt. %, the thickeners in an amount from about 0.1 to 30 wt. % and the hair-care agents at a concentration of about 0.1 to 5.0 wt. %.

Moreover, the colorant can contain additional common additives, for example antioxidants such as ascorbic acid, thioglycolic acid or sodium sulfite, as well as perfume oils, penetrants, buffer systems, complexing agents, preservatives, wetting agents, emulsifiers, thickeners and hair-care agents.

The ready-to-use colorant of the invention is prepared just before use by mixing the dye carrier composition containing the dyes with an oxidant.

Suitable oxidants are mainly hydrogen peroxide or its products of addition to urea, melamine, sodium borate or sodium carbonate, in the form of a 1-12%, preferably 3-6% aqueous solution. The weight ratio of dye carrier composition to oxidant is preferably from about 5:1 to 1:3, and particularly from 1:1 to 1:2. Larger amounts of oxidant are used primarily at higher concentrations of oxidative dye precursors in the hair colorant or when stronger bleaching of the keratin fibers (particularly hair) is wanted at the same time.

The pH of the ready-to-use colorant obtained by mixing the dye carrier composition with the oxidant assumes a value which depends on the pH values of the dye carrier composition and of the oxidant and on the mixing ratio. The ready-to-use colorant has a basic pH greater than 7 and preferably one between 8 and 11. The adjustment to a basic pH is preferably done with ammonia, although an organic amine, for example 2-amino-2-methyl-1 -propanol,tris(hydroxymethyl)aminomethane, monoethanolamine or tri-ethanolamine, or a mixture of an organic amine and ammonia can also be used, as can an inorganic base such as sodium hydroxide or potassium hydroxide. If the pH is too high, it can be corrected with an inorganic or organic acid, for example phosphoric acid, acetic acid, lactic acid, ascorbic acid, citric acid or tartaric acid.

An amount of this mixture sufficient for the coloring treatment, in general about 60 to 200 grams, is then applied to the keratin fibers, and the mixture is allowed to act on the keratin fibers at about 15 to 50° C., preferably at 30 to 40° C., for about 10 to 45 min and preferably for 30 min, after which the keratin fibers are rinsed with water and dried. Optionally, after this rinsing the fibers are washed with a shampoo and possibly post-rinsed with a weak organic acid, for example citric acid or tartaric acid. The keratin fibers are then dried.

It is also possible, when coloring hair that has been damaged to varying degrees (for example when post-coloring previously oxidatively died hair areas), to apply the dye carrier composition to the previously damaged hair parts (for example hair tips) without the oxidant—either as is or diluted only with an another acidic, neutral or basic aqueous component, while applying the mixture of dye carrier composition and oxidant to the hair parts (for example to the hair roots and the hair shafts) that have previously been damaged only slightly or not at all. The aqueous component used for dilution can contain the additives commonly employed for solutions, creams, emulsions or gels. By this method, it is possible to achieve colorations characterized by a balance between roots and tips that is gentle to the hair and is adapted to the nature of the hair. This is not possible when common oxidative hair colorants are used, because the coupling of the dye precursors always requires an oxidant.

The colorant of the invention gives rise to colorations covering the entire color spectrum and which are characterized, in particular, by an unusual color intensity and brightness, good color balance between damaged and undamaged hair (for example between hair tips and new hair growth) and their marked gentleness to the hair.

The following examples will explain the subject matter of the invention in greater detail without limiting its scope.

### EXAMPLES

### Examples 1 to 11

### Hair Colorants

| | |
|---|---|
| Dye of formula (I), (II) and/or (III) | as per Table 1 |
| Steareth-20 | 2.8 g |
| Cetylstearyl alcohol | 12.0 g |
| Ethanol | 20.0 g |
| Water, demineralized | to 100.00 g |

The pH was adjusted to 10 with 25% ammonia.

5 of the foregoing dye carrier composition was mixed with 7.5 g of a 9% hydrogen peroxide solution. The resulting ready-to-use hair colorant was applied to small hair strands and uniformly distributed with a brush. After an exposure time of 30 min at 40° C., the hair was rinsed with luke-warm water, washed with a shampoo, rinsed with luke-warm water and then dried. The coloring results are collected in the following Table 1.

L*a*b* color values given in the examples were determined with a Chromameter II color-measuring instrument supplied by Minolta.

The L-value stands for brightness (namely, the lower the L-value the higher is the color intensity), whereas the a-value is a measure of the red content (namely, the higher the a-value the higher is the red content). The b-value is a measure of the blue content of the color, the blue content being the higher the more negative the b-value.

Unless otherwise indicated, all percentages given in the present patent application are by weight.

TABLE 1

| | | | | Color | | |
|---|---|---|---|---|---|---|
| Example | | Color Shade | | Values of the Hair Strands | | |
| No. | Direct Dye(s) | After Dyeing | | L | a | b |
| 1 | 3 (or 5)-{[4-(benzylmeth- | red | Before dyeing: | +83.30 | −0.48 | +10.40 |
| | ylamino)phenyl]azo}- | | After dyeing: | +27.38 | +57.26 | +16.73 |
| | 1,2-(or 1,4)-dimethyl- | | | | | |
| | 1,2,4-triazolium bromide | | | | | |
| | (Basic Red 46): 1.0 g | | | | | |
| 2 | N,N-dimethyl-3-{[4- | blue | Before dyeing: | +83.30 | −0.48 | +10.40 |
| | (methylamino)-9,10- | | After dyeing: | +21.01 | +19.43 | −37.57 |
| | diketo-9,10-dihydro- | | | | | |
| | 1-anthracenyl]amino}- | | | | | |
| | N-propyl-1-propan- | | | | | |
| | aminium chloride: 1.0 g | | | | | |
| 3 | 3-[(4,5-dihydro-3-methyl- | yellow | Before dyeing: | +83.30 | −0.48 | +10.40 |
| | 5-keto-1-phenyl-1H-pyra- | | After dyeing: | +70.17 | +9.36 | +73.34 |
| | zol-4-yl)azo]-N,N,N-tri- | | | | | |
| | methylbenzenaminium- | | | | | |
| | chloride (Basic Yellow | | | | | |
| | 57): 1.3 g | | | | | |
| 4 | 3-[(4,5-dihydro-3-methyl- | green | Before dyeing: | +83.30 | −0.48 | +10.40 |
| | 5-keto-1-phenyl-1H-pyra- | | After dyeing: | +37.58 | −19.99 | +7.79 |
| | zol-4-yl)azo]-N,N,N-tri- | | | | | |
| | methylbenzenaminium- | | | | | |
| | chloride (Basic Yellow | | | | | |
| | 57): 0.92 g | | | | | |
| | N,N-dimethyl-3-{[4-(methyl- | | | | | |
| | amino)-9,10-diketo-9,10- | | | | | |
| | dihydro-1-anthracenyl]amino}- | | | | | |
| | N-propyl-1-propanaminium | | | | | |
| | chloride: 0.3 g | | | | | |
| 5 | 3-[(4,5-dihydro-3-methyl- | wine-red | Before dyeing: | +83.30 | −0.48 | +10.40 |
| | 5-keto-1-phenyl-1H-pyra- | | After dyeing: | +18.40 | +5.38 | −0.67 |
| | zol-4-yl)azo]-N,N,N-tri- | | | | | |
| | methylbenzenaminium | | | | | |
| | chloride (Basic Yellow | | | | | |
| | 57): 0.43 g | | | | | |
| | N,N-dimethyl-3-{[4-(methyl- | | | | | |
| | amino)-9,10-diketo-9,10- | | | | | |
| | dihydro-1-anthracenyl]amino}- | | | | | |
| | N-propyl-1-propanaminium | | | | | |
| | bromide: 0.35 g | | | | | |
| | 3 (or 5)-{[4-(benzylmethyl- | | | | | |
| | amino)phenyl]azo}-1,2- | | | | | |
| | (or 1,4)-dimethyl-1,2,4- | | | | | |
| | triazolium bromide (Basic | | | | | |
| | Red 46): 0.33 g | | | | | |
| 6 | 3-[(4,5-dihydro-3-methyl- | dark-brown | Before dyeing: | +83.30 | −0.48 | +10.40 |
| | 5-keto-1-phenyl-1H-pyra- | | After dyeing: | +20.94 | +4.63 | −1.16 |
| | zol-4-yl)azol-N,N,N-tri- | | | | | |
| | methylbenzenaminium- | | | | | |
| | chloride (Basic Yellow | | | | | |
| | 57): 0.72 g | | | | | |
| | N,N-dimethyl-3-{[4-(methyl- | | | | | |
| | amino)-9,10-diketo-9,10- | | | | | |
| | dihydro-1-anthracenyl]amino)- | | | | | |
| | N-propyl-1-propanaminium | | | | | |
| | chloride: 0.33 g | | | | | |
| | 3 (or 5)-{[4-(benzylmethyl- | | | | | |
| | amino)phenyl]azo}-1,2- | | | | | |
| | (or 1,4)-dimethyl-1,2,4-tri- | | | | | |
| | azolium bromide (Basic Red 46): | | | | | |
| | 0.13 g | | | | | |
| 7 | 3-[(4,5-dihydro-3-methyl- | medium- | Before dyeing: | +83.30 | −0.48 | +10.40 |
| | 5-keto-1-phenyl-1H-pyra- | brown | After dyeing: | +21.83 | +5.54 | +3.00 |
| | zol-4-yl)azo]-N,N,N-tri- | | | | | |
| | methylbenzenaminium- | | | | | |
| | chloride (Basic Yellow | | | | | |
| | 57): 1.65 g | | | | | |
| | N,N-dimethyl-3-{[4-(methyl- | | | | | |
| | amino)-9,10-diketo-9,10- | | | | | |
| | dihydro-1-anthracenyl]amino}- | | | | | |
| | N-propyl-1-propanaminium | | | | | |
| | chloride: 0.4 g | | | | | |
| | 3 (or 5)-{[4-(benzylmethyl- | | | | | |
| | amino)phenyl]azo}-1,2-(or 1,4)- | | | | | |
| | dimethyl-1,2,4-triazolium | | | | | |
| | bromide (Basic Red 46): 0.4 g | | | | | |
| 8 | 3-((4,5-dihydro-3-methyl-5-keto-1-phenyl-1H- | medium- | | | | |
| | pyrazol-4-yl)azol-N,N,N-trimethylbenzen- | brown | | | | |
| | aminium chloride (Basic Yellow 57); 1.65 g | | | | | |
| | N,N-dimethyl-3-{[4-(methylamino)-9,10- | | | | | |
| | diketo-9,10-dihydro-1-anthracenyl]amino}- | | | | | |
| | N-propyl-1-propanaminium chloride: 0.4 g | | | | | |
| | 2-{[4-(dimethylamino)phenyl]azo}- | | | | | |
| | 1,3-dimethyl-1H-imidazol-3-ium chloride (Basic | | | | | |
| | Red 51): 0.27 g | | | | | |
| 9 | 3-[(4,5-dihydro-3-methyl-5-keto-1-phenyl- | medium- | | | | |
| | 1H-pyrazol-4-yl)azo]-N,N,N-trimethylbenzen- | brown | | | | |
| | aminium chloride (Basic Yellow 57): 1.50 g | | | | | |
| | 1-methyl-4-{[methyl{4-methoxyphenyl)hydra- | | | | | |
| | zono]methyl}pyridinium methylsulfate | | | | | |
| | (Basic Yellow 91): 0.15 g | | | | | |
| | N,N-dimethyl-3-{[4-(methylamino)-9,10- | | | | | |
| | diketo-9,10-dihydro-1-anthracenyl]amino}- | | | | | |
| | N-propyl-1-propanaminium chloride: 0.4 g | | | | | |
| | 2-{[4-(dimethylamino)phenyl]azo}- | | | | | |
| | 1,3-dimethyl-1H-imidazol-3-ium chloride (Basic | | | | | |
| | Red 51): 0.27 g | | | | | |
| 10 | 3-[(4,5-dihydro-3-methyl-5-keto-1- | dark- | | | | |
| | phenyl-1H-pyrazol-4-yl)azo]-N,N,N-trimethyl- | brown | | | | |
| | benzenaminium chloride (Basic Yellow 57): 0.72 g | | | | | |
| | N,N-dimethyl-3-{[4-(methylamino)-9,10- | | | | | |
| | diketo-9,10-dihydro-1-anthracenyl]amino}- | | | | | |
| | N-propyl-1-propanaminium chloride: 0.33 g | | | | | |
| | 2-{[4-(dimethylamino)phenyl]azo)-1,3- | | | | | |
| | dimethyl-1H-imidazol-3-ium chloride (Basic | | | | | |
| | Red 51): 0.10 g | | | | | |
| 11 | 3-[(4,5-dihydro-3-methyl-5-keto-1-phenyl- | dark- | | | | |
| | 1H-pyrazol-4-yl)azo]-N,N,N-trimethylbenzen- | brown | | | | |
| | aminium chloride (Basic Yellow 57): 0.72 g | | | | | |
| | N,N-dimethyl-3-{[4-(methylamino)-9,10- | | | | | |
| | diketo-9,10-dihydro-1-anthracenyl]amino}- | | | | | |
| | N-propyl-1-propanaminium chloride: 0.33 g | | | | | |
| | 1,4-dimethyl-5-{[4-(dimethylamino)pheny]azo}- | | | | | |
| | 1,2,4-triazolium chloride (C.I. 11055; | | | | | |
| | Basic Red 22): 0.12 g | | | | | |

1. Ready-to-use colorant for brightening and coloring keratin fibers, characterized in that it contains (a) an oxidant and (b) at least one yellow direct dye of formula (I) and/or a red direct dye of formula (II) and/or a blue direct dye of general formula (III) and that c) it has a basic pH 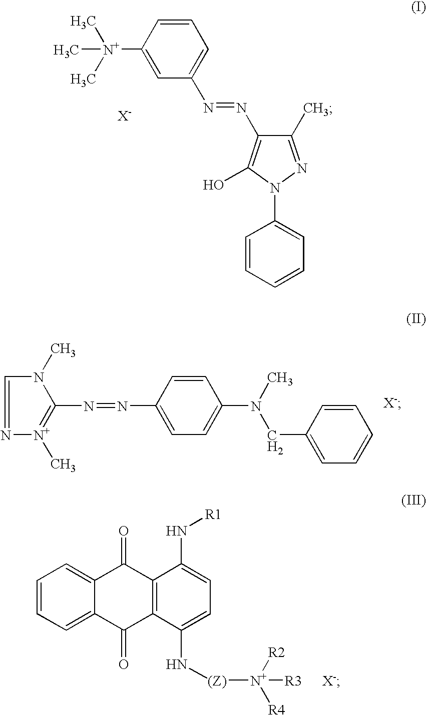
wherein
R1 denotes a hydrogen atom, a (C₁-C₄)-alkyl group, a phenyl group or a (C₂-C₄)hydroxyalkyl group,
R2, R3 and R4 can be equal or different and denote a (C₁-C₄)-alkyl group,
Z denotes a (C₂-C₈)-alkylene diradical and
X⁻ denotes an anion from the group consisting of chloride, bromide, alkylsulfate, arylsulfate, sulfate and acetate.

2. Ready-to-use colorant for brightening and coloring keratin fibers, characterized in that it contains (a) an oxidant, (b) at least one blue direct dye of general formula (III) and c) at least one yellow direct dye of formula (I), and/or at least one red direct dye of formula (II) and (d) that it has a basic pH 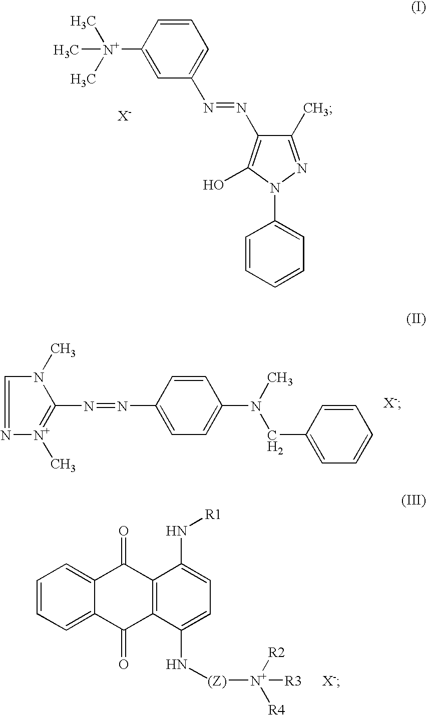
wherein
R1 denotes a hydrogen atom, a (C₁-C₄)-alkyl group, a phenyl group or a (C₂-C₄)-hydroxyalkyl group,
R2, R3 and R4 can be equal or different and denote a (C₁-C₄)-alkyl group,
Z denotes a (C₂-C₆)-alkylene diradical and
X⁻ denotes an anion from the group consisting of chloride, bromide, alkylsulfate, arylsulfate, sulfate and acetate.

3. Colorant according to claim 1 or 2, characterized in that the dye of formula (III) is selected from among compounds of formula (III) wherein R1 denotes a (C₁-C₄)-alkyl group, the R2 and R3 groups denote a methyl group and R4 stands for a (C₁-C₄)-alkyl group;
Z denotes a C₂- or C₃-alkylene diradical and
X⁻ denotes an anion from the group consisting of chloride, bromide and alkylsulfate.

4. Colorant according to claim 1 or 2, characterized in that the dye of formula (I) is 3-[(4,5-dihydro-3-methyl-5-keto-1-phenyl-1H-pyrazol-4-yl)azo]-N,N,N-trimethyl-benzenaminium chloride or 3-[(3-methyl-5-hydroxy-1-phenyl-1H-pyrazol-4-yl)azo]trimethylammoniobenzene chloride.

5. Colorant according to claim 1 or 2, characterized in that the dye of formula (II) is 3 (or 5)-{[4-(benzylmethylamino)phenyl]azo}-1,2-(or 1,4)-dimethyl-1,2,4-triazolium bromide.

6. Colorant according to one of claims 1 to 3, characterized in that the dye of formula (III) is selected from among N,N,N-trimethyl-3-{[4-(methylamino)-9,10-dike-to-9,10-dihydro-1-anthracenyl]amino}-1-propanaminium methylsulfate, N,N-dimethyl-3-{[4-(methylamino)-9, 10-diketo-9,10-dihydro-1-anthracenyl]amino}-N-propyl-1-propanaminium chloride and N,N-dimethyl-3-{[4-(methylamino)-9,10-diketo-9,10-dihydro-1-anthracenyl]amino}-N-propyl-1-propanaminium bromide.

7. Colorant according to one of claims 1 to 6, characterized in that it contains the dyes of formulas (I) to (III) in a total amount from 0.001 to 8 wt. %.

8. Colorant according to one of claims 1 to 7, characterized in that besides the dyes of formulas (I) to (III) it contains additional direct and/or oxidative dye precursors.

9. Colorant according to one of claims 1 to 8, characterized in that the ready-to-use colorant has a pH from 8 to 11.

10. Two-component colorant for coloring keratin fibers, consisting of a dye carrier composition (A) and a component (B) containing an oxidant, characterized in that the dye carrier composition (A) contains at least one yellow direct dye of formula (I) and/or a red direct dye of formula (II) and/or a blue direct dye of general formula (III) and optionally other direct and/or oxidative dye precursors 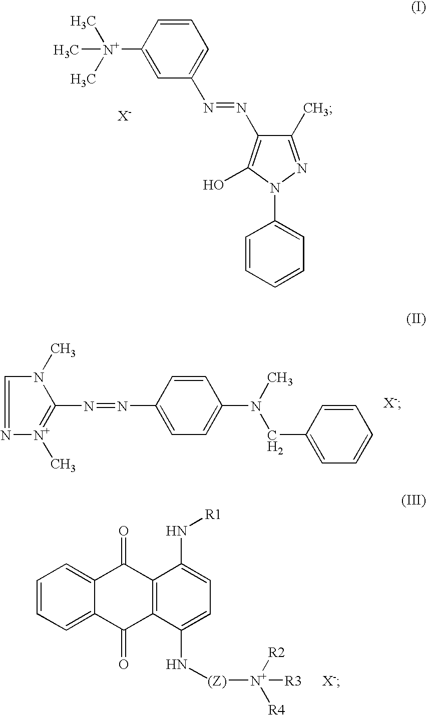
wherein
R1 denotes a hydrogen atom, a (C₁-C₄)-alkyl group, a phenyl group or a (C₂-C₄)-hydroxyalkyl group,
R2, R3 and R4 can be equal or different and denote a (C₁-C₄)-alkyl group,
Z denotes a (C₂-C₆)-alkylene diradical and
X⁻ denotes an anion from the group consisting of chloride, bromide, alkylsulfate, arylsulfate, sulfate and acetate.

11. Colorant according to claim 10, characterized in that the dye of formula (I) is 3-[(4,5-dihydro-3-methyl-5-keto-1-phenyl-1H-pyrazol-4-yl)azo]-N,N,N-trimethylbenzenaminium chloride or 3-[(3-methyl-5-hydroxy-1-phenyl-1H-pyrazol-4-yl)azo]-trimethylammoniobenzene chloride,

12. Colorant according to claim 10, characterized in that the dye of formula (II) is 3 (or 5)-{[4-(benzylmethylamino)phenyl]azo}-1,2-(or 1,4)-dimethyl-1,2,4-triazolium bromide.

13. Colorant according to claim 10, characterized in that the dye of formula (III) is selected from among N,N,N-trimethyl-3-{[4-(methylamino)-9,10-diketo-9,10-dihydro-1-anthracenyl]amino}-1-propanaminium methylsulfate, N,N-dimethyl-3-{[4-(methylamino)-9,10-diketo-9,10-dihydro-1-anthracenyl]amino}-N-propyl-1-propanaminium chloride and N,N-dimethyl-3-{[4-(methylamino)-9,10-diketo-9,10-dihydro-1 -anthracenyl]amino}-N-propyl-1-propanaminium bromide.

14. Colorant according to one of claims 1 to 13, characterized in that it is a hair colorant.

15. Method for coloring keratin fibers whereby an amount of the colorant sufficient for the coloring is applied to the hair, and after an exposure time of 10 to 45 minutes at 15 to 50° C. the hair is rinsed with water and optionally dried, characterized in that a colorant according to one of claims 1 to 14 is used.

16. Method for coloring keratin fibers, characterized in that in a first step an oxidant-free colorant (dye carrier composition) containing at least one yellow direct dye of formula (I) and/or a red direct dye of formula (II) and/or a blue direct dye of general formula (III) and optionally other dyes 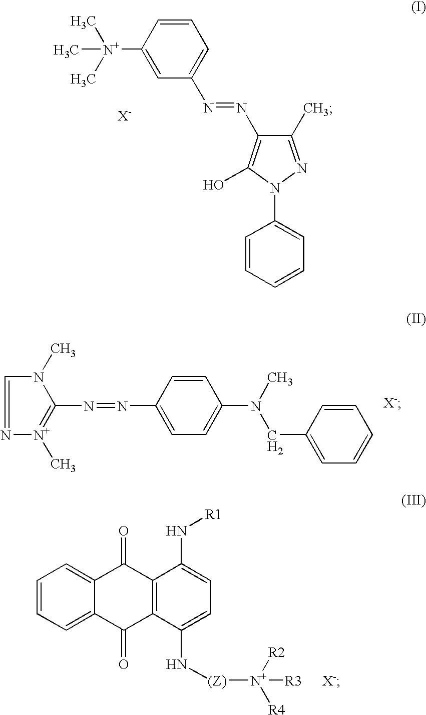
wherein
R1 denotes a hydrogen atom, a (C₁-C₄)-alkyl group, a phenyl group or a (C₂-C₄)-hydroxyalkyl group,
R2, R3 and R4 can be equal or different and denote a (C₁-C₄)-alkyl group,
Z denotes a (C₂-C₈)-alkylene diradical and
X⁻ denotes an anion from the group consisting of chloride, bromide, alkylsulfate, arylsulfate, sulfate and acetate,
is applied to parts of the hair previously damaged by oxidants, and in a second step the aforesaid dye carrier composition is first mixed with an oxidant and then applied to the remainder of the hair.

17. Method according to claim 15 or 16, characterized in that the dye carrier composition is mixed with the oxidant in a weight ratio from 5:1 to 1:3.
